(19) United States
(12) Patent Application Publication
(10) Pub. No.: US 2024/0399117 A1
(43) Pub. Date: Dec. 5, 2024

(54) SYSTEMS, METHODS, AND DEVICES FOR ADVANCING AN ENDOVASCULAR INSTRUMENT

(71) Applicant: Remedy Robotics, Inc., San Francisco, CA (US)

(72) Inventors: Jake Anthony Sganga, San Francisco, CA (US); David James Bell, Mill Valley, CA (US)

(21) Appl. No.: 18/676,954

(22) Filed: May 29, 2024

Related U.S. Application Data
(60) Provisional application No. 63/504,943, filed on May 30, 2023.

(51) Int. Cl. A61M 25/09; A61M 25/00; A61M 25/01

(52) U.S. Cl. CPC A61M 25/09041; A61M 25/0023; A61M 25/01; A61M 2025/0042; A61M 2025/0177

(57) ABSTRACT

A system for advancing a guidewire through a patient lumen including a first advancement device and a second advancement device. The second advancement device may be positioned between the first advancement device and an access point to the patient lumen. The first advancement device may close to contact the guidewire and open to not contact the wire. When the first advancement device is closed the first advancement device may move from a first position to a second position to advance the guidewire through the patient lumen. The first advancement device may rotate to rotate the guidewire. The second advancement device may be coupled to a microcatheter.

### PRIORITY APPLICATION

This application claims priority to U.S. Provisional Patent Application No. 63/504,943, filed May 30, 2023. Any and all applications for which a foreign or domestic priority claim is identified in the Application Data Sheet as filed with the present application are hereby incorporated by reference under 37 CFR 1.57.

### BACKGROUND

### Field

Some embodiments of the present disclosure are direction to systems, methods, and device for robotically advancing endovascular instruments, such as guidewires, coils, stents, catheters and the like.

### Description

Endovascular medical procedures are common. During an endovascular procedure, a tool or medical instrument that is generally configured as a long, thin, flexible body is inserted into and navigated through a lumen or other cavity of the body.

In some instances, the tools or medical instruments are articulable or controllable, for example, using one or more pull wires, to allow an operator to navigate the tool or medical instrument within the body. Such navigation is often accomplished through deflection (for example, bending) of the distal tip of the tool or medical instrument.

Some tools or medical instruments are configured for manual control, for example, using knobs or levers mounted on a proximally-located handle of the tool or medical instrument. In other instances, the tools or medical instruments can be configured for robotic control, for example, control by a robotic medical system. In some embodiments, an operator can use the robotic medical system (for example, a controller, user interface, and/or the like) to robotically control the tool or medical instrument.

### BRIEF DESCRIPTION OF THE DRAWINGS

Various embodiments are depicted in the accompanying drawings for illustrative purposes and should in no way be interpreted as limiting the scope of the embodiments. It is to be understood that the attached drawings are for the purpose of illustrating concepts disclosed in the present application and may not be to scale. Furthermore, various features of different disclosed embodiments can be combined to form additional embodiments, which are part of this disclosure.

FIGS. 1A-1B illustrate an example system for advancing a guidewire through a patient lumen.

FIGS. 2A-2C illustrate another system for advancing a guidewire through a patient lumen according to some embodiments described herein.

FIGS. 3A-3B illustrate another system for advancing a guidewire through a patient lumen according to some embodiments described herein.

FIGS. 4A-4B illustrate another system for advancing a guidewire through a patient lumen according to some embodiments described herein.

FIGS. 5A-5D illustrate various views of a first tool advancement device according to some embodiments described herein.

FIGS. 5E-5H illustrate various views of a second tool advancement device according to some embodiments described herein.

FIG. 6 illustrates a collet according to some embodiments described herein.

FIG. 7 is a block diagram illustrating an example embodiment of a computer system configured to run software for implementing one or more embodiments of the systems, methods, and devices disclosed herein.

### DETAILED DESCRIPTION

Although several embodiments, examples, and illustrations are disclosed below, it will be understood by those of ordinary skill in the art that the system, methods, and devices described herein extend beyond the specifically disclosed embodiments, examples, and illustrations and includes other uses of the system, methods, and devices and obvious modifications and equivalents thereof. Embodiments of the disclosure are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the disclosure. In addition, embodiments of the disclosure can comprise several novel features and no single feature is solely responsible for its desirable attributes or is essential to practicing the system, methods, and devices herein described.

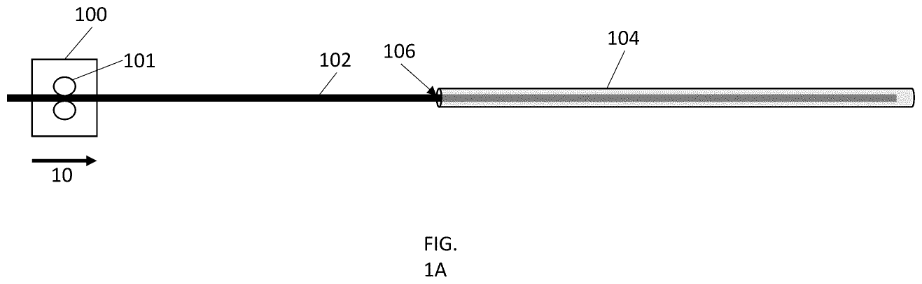

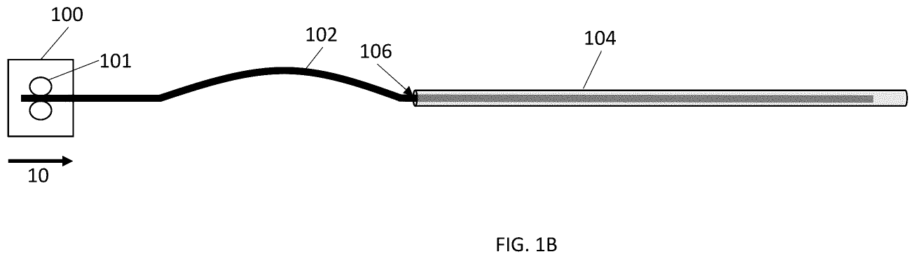

An endovascular tool 102, for example, a guidewire, may be inserted into a lumen 104 via a remotely controlled system 100, as shown in FIG. 1A. The lumen 104 can be, for example, a lumen formed through another endovascular tool such as a catheter or a patient lumen. The endovascular tool 102 be inserted into the lumen 104 through an access point 106. In order to advance the endovascular tool 102 through the patient lumen 104, the system 100 may apply an axial force 10 to the endovascular tool 102 at a point of application 12 outside of the lumen 104. In some instances, the system 100 may include rollers 101 configured to apply the axial force 10 to the endovascular tool 102. The rollers 101 may be configured to rotate in order to apply the axial force 10 to the endovascular tool 102. In other embodiments, rollers 101 may be replaced with a clamping mechanism that clamps onto the endovascular tool and then moves backwards and forwards to advance the endovascular tool 102.

In order to navigate through the anatomical luminal networks, the endovascular tool 102 may be include a small diameter, for example, between about 0.010 inches and about 0.038 inches, and may be generally flexible. Accordingly, when the system 100 applies the axial force 10 to the endovascular tool 102, the endovascular tool 102 may deform, bend, or buckle between a point of application 12 of the axial force 10 and the access point 106, as shown in FIG. 1B. Additionally, when the rollers 101 rotate, due to the small diameter of the endovascular tool 102, the rollers 101 may slip and not apply the axial force 10 to the endovascular tool 102 preventing or inhibiting the system 100 from precisely controlling advancement of the endovascular tool 102. These problems can be overcome using the systems described below.

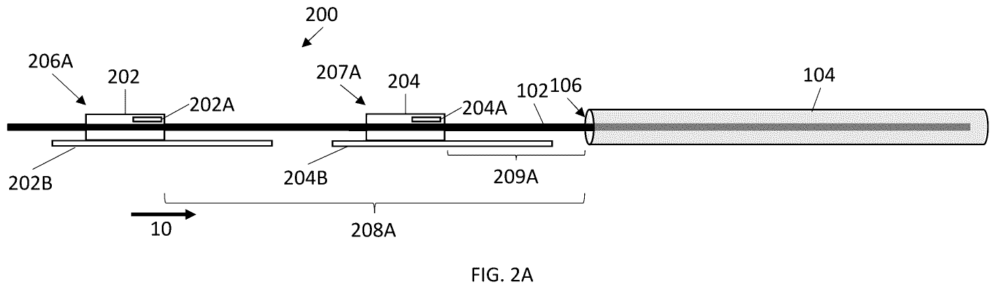

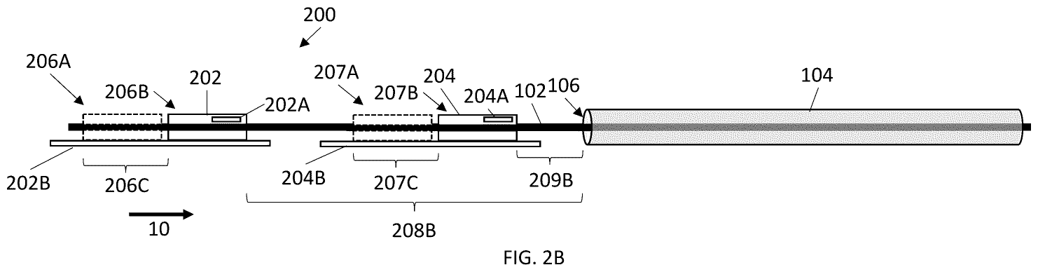

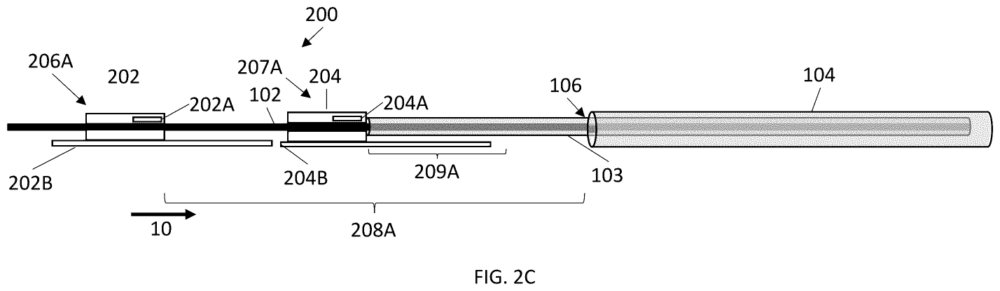

FIGS. 2A-2C show a system 200 for advancing an endovascular tool 102 through a patient lumen 104. In some embodiments, the endovascular tool 102 may be advanced through the patient lumen 104 with a microcatheter 103, as shown in FIG. 2C. In some embodiments, the system 200 may include a first tool advancement device 202 and a second tool advancement device 204. In some embodiments, the second tool advancement device 204 may be positioned between the first tool advancement device 202 and an access point 106 to the patient lumen 104. In some embodiments, the first tool advancement device 202 may be positioned proximal to the second tool advancement device 204 and/or the access point 106. In some embodiments, the second tool advancement device 204 may be positioned distal to the first tool advancement device 202 and/or proximal to the access point 106.

In some embodiments, the first tool advancement device 202 and/or the second tool advancement device 204 may be configured to move (e.g., open/close) between an open configuration and a closed configuration. In some embodiments, the first tool advancement device 202 may include an actuator 202A configured to move the first tool advancement device 202 between the open configuration and the closed configuration, and the second tool advancement device 204 may include an actuator 204A configured to move the second tool advancement device 204 between the open configuration and the closed configuration.

In some embodiments, when the first tool advancement device 202 is in the closed configuration, the first tool advancement device 202 may contact the endovascular tool 102 and/or apply a force to the endovascular tool 102. In some embodiments, when the first tool advancement device 202 is in the open configuration, the first tool advancement device 202 may not contact the endovascular tool 102.

In some embodiments, when the second tool advancement device 204 is in the closed configuration, the second tool advancement device 204 may contact the endovascular tool 102 and/or apply the force to the endovascular tool 102, as shown in FIGS. 2A and 2B. In some embodiments, when the second tool advancement device 204 is in the open configuration, the second tool advancement device 204 may not contact the endovascular tool 102 and/or the microcatheter 103.

In some embodiments, the first tool advancement device 202 may be configured to move between a first position 206A and a second position 206B. When the first tool advancement device 202 is positioned at the first position 206A, the first tool advancement device 202 may be positioned a first distance 208A from the access point 106. When the first tool advancement device 202 is positioned at the second position 206B, the first tool advancement device 202 may be positioned a second distance 208B from the access point 106. In some embodiments, the first distance 208A may be larger than the second distance 208B. Accordingly, when the first tool advancement device 202 is positioned at the first position 206A, the first tool advancement device 202 may be further away from the access point 106 than when the first tool advancement device 202 is positioned at the second position 206B. The first tool advancement device 202 may move away (i.e., further) from the access point 106 when the first tool advancement device 202 moves from the second position 206B to the first position 206A. The first tool advancement device 202 may move towards (i.e., closer to) the access point 106 when the first tool advancement device 202 moves from the first position 206A to the second position 206B.

In some embodiments, the first tool advancement device 202 may include or be mounted on a rail or linear movement system 202B. The first tool advancement device 202 may translate along the rail system 202B to move between the first position 206A and the second position 206B.

In some embodiments, the first tool advancement device 202 may be configured to advance the endovascular tool 102 through the patient lumen 104. In some embodiments, if the first tool advancement device 202 is not positioned at the first position 206A, the first tool advancement device 202 may move to the first position 206A. The first tool advancement device 202 may be in the open configuration when the first tool advancement device 202 moves to the first position 206A so the first tool advancement device 202 does not contact the endovascular tool 102 and/or retract the endovascular tool 102 from the patient lumen 104. In some embodiments, the first tool advancement device 202 may move from the closed configuration to the open configuration before the first tool advancement device 202 moves to the first position 206A. In some embodiments, the actuator 202A may move the first tool advancement device 202 from the closed configuration to the open configuration.

In some embodiments, the first tool advancement device 202 may move from the open configuration to the closed configuration when the first tool advancement device 202 is positioned at the first position 206A. In some embodiments, the actuator 202A may move the first tool advancement device 202 from the open configuration to the closed configuration.

In some embodiments, the first tool advancement device 202 may move from the first position 206A to the second position 206B when the first tool advancement device 202 is in the closed configuration to advance the endovascular tool 102 through the patient lumen 104, as shown in FIG. 2B. In some embodiments, the first tool advancement device 202 may advance the endovascular tool 102 a distance through the patient lumen 104. In some embodiments, the distance may include a distance 206C between the first position 206A and the second position 206B. In some embodiments, the distance 206C may include a distance of about 1 mm, 2 mm, 3 mm, 4 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, and/or any value between the aforementioned values. In some embodiments, the distance 206C may include a distance between about 10 mm and about 70 mm. In some embodiments, the distance 206C may include a distance between about 20 mm and about 60 mm. In some embodiments, the distance 206C may include a distance between about 30 mm and about 50 mm.

In some embodiments, the first tool advancement device 202 may apply the axial force 10 to the endovascular tool 102 via friction between the first tool advancement device 202 and the endovascular tool 102 generated by the force applied to the endovascular tool 102 by the first tool advancement device 202. The axial force 10 applied to the endovascular tool 102 by the first tool advancement device 202 may cause the endovascular tool 102 to advance through the patient lumen 104.

In some embodiments, after the first tool advancement device 202 moves to the second position 206B, the first tool advancement device 202 may move from the closed configuration to the open configuration and/or the actuator 202A may move the first tool advancement device 202 from the closed configuration to the open configuration. In some embodiments, after the first tool advancement device 202 moves from the closed configuration to the open configuration, the first tool advancement device 202 may move from the second position 206B to the first position 206A, as described above.

In some embodiments, the second tool advancement device 204 may contact the endovascular tool 102 when the second tool advancement device 204 is in the closed configuration, as shown in FIG. 2A. The second tool advancement device 204 may be in the closed configuration when the first tool advancement device 202 moves from the second position 206B to the first position 206A (e.g., away from the second tool advancement device 204). In some embodiments, if the second tool advancement device 204 is in the open configuration when the first tool advancement device 202 is in the second position 206B, the second tool advancement device 204 may move to the closed configuration and/or the actuator 204A may move the second tool advancement device 204 to the closed configuration. In some embodiments, the second tool advancement device 204 may move to the closed configuration and/or the actuator 204A may move the second tool advancement device 204 to the closed configuration before the first tool advancement device 202 moves from the second position 206B to the first position 206A.

In some embodiments, as described above, the second tool advancement device 204 may apply the force to the endovascular tool 102 when the second tool advancement device 204 is in the closed configuration. When the first tool advancement device 202 moves from the second position 206B to the first position 206A, if the first tool advancement device 202 contacts the endovascular tool 102, the second tool advancement device 204 may prevent or inhibit the first tool advancement device 202 from removing the endovascular tool 102 from the patient lumen 104. In some embodiments, friction between the second tool advancement device 204 and the endovascular tool 102 may prevent or inhibit the first tool advancement device 202 from removing the endovascular tool 102 from the patient lumen 104.

In some embodiments, the second tool advancement device 204 may move from the closed configuration to the open configuration and/or the actuator 204A may move the second tool advancement device 204 from the closed configuration to the open configuration when the first tool advancement device 202 is in the first position. In some embodiments, after the first tool advancement device 202 moves to the first position 206A, the second tool advancement device 204 may move from the closed configuration to the open configuration and/or the actuator 204A may move the second tool advancement device 204 from the closed configuration to the open configuration.

In some embodiments, as shown in FIG. 2C, the endovascular tool 102 may be advanced through the patient lumen 104 in or through a microcatheter 103.

In some embodiments, as shown for example in FIG. 2B, the second tool advancement device 204 may be configured to move between a first position 207A and a second position 207B. When the second tool advancement device 204 is positioned at the first position 207A, the second tool advancement device 204 may be positioned a first distance 209A from the access point 106. When the second tool advancement device 204 is positioned at the second position 207B, the second tool advancement device may be positioned a second distance 209B from the access point 106. In some embodiments, the first distance 209A may be larger than the second distance 209B. Accordingly, when the second tool advancement device 204 is positioned at the first position 207A, the second tool advancement device 204 may be further away from the access point 106 than when the second tool advancement device 204 is positioned at the second position 207B. The second tool advancement device 204 may move away (i.e., further) from the access point 106 when the second tool advancement device 204 moves from the second position 207B to the first position 207A. The second tool advancement device 204 may move towards (i.e., closer to) the access point 106 when the second tool advancement device 204 moves from the first position 207A to the second position 207B.

In some embodiments, a distance 207C between the first position 207A of the second tool advancement device 204 and the second position 207B of the second tool advancement device 204 may include a distance longer than the distance 206C between the first position 206A of the first tool advancement device 202 and the second position 206B of the first tool advancement device 202. In some embodiments, the distance 207C may include a distance shorter than the distance 206C. In some embodiments, the distance 207C may include a same distance as the distance 206C.

In some embodiments, the second tool advancement device 204 may include a rail system or linear movement system 204B. The second tool advancement device 204 may translate along the rail system 204B to move between the first position 207A and the second position 207B.

In some embodiments, the second tool advancement device 204 may be configured to move from the first position 207A to the second position 207B to advance the endovascular tool 102 though the patient lumen 104, as shown in FIG. 2B. In some embodiments, the second tool advancement device 204 may be configured to move from the first position 207A to the second position 207B to advance the microcatheter 103 through the patient lumen 104.

In some embodiments, the second tool advancement device 204 may be configured to move from the first position 207A to the second position 207B when the first tool advancement device 202 moves from the first position 206A to the second position 206B. In some embodiments, as shown in FIG. 2B, the first tool advancement device 202 and the second tool advancement device 204 may advance the endovascular tool 102 through the patient lumen 104.

In some embodiments, when the second tool advancement device 204 is positioned at the first position 207A, the second tool advancement device 204 may be in the closed configuration. If the second tool advancement device 204 is in the open configuration, the second tool advancement device 204 may move to the closed configuration and/or the actuator 204A may move the second tool advancement device 204 to the closed configuration. In some embodiments, the second tool advancement device 204 may move from the first position 207A to the second position 207B when the second tool advancement device 204 is in the closed configuration.

In some embodiments, the second tool advancement device 204 may apply the axial force 10 to the endovascular tool 102 via friction between the second tool advancement device 204 and the endovascular tool 102 generated by the force applied to the endovascular tool 102 by the second tool advancement device 204. The axial force 10 applied to the endovascular tool 102 by the second tool advancement device 204 may cause the endovascular tool 102 to advance through the patient lumen 104.

In some embodiments, the second tool advancement device 204 may apply the axial force 10 to the microcatheter 103 via friction between the second tool advancement device 204 and the microcatheter 103 generated by the force applied to the microcatheter 103 by the second tool advancement device 204. The axial force 10 applied to the microcatheter 103 by the second tool advancement device 204 may cause the microcatheter 103 to advance through the patient lumen 104.

In some embodiments, after the second tool advancement device 204 moves to the second position 207B, the second tool advancement device 204 may move from the closed configuration to the open configuration and/or the actuator 204A may move the second tool advancement device 204 from the closed configuration to the open configuration. In some embodiments, the after the second tool advancement device 204 moves from the closed configuration to the open configuration, the second tool advancement device 204 may move from the second position 207B to the first position 207A.

In some embodiments, the first tool advancement device 202 and/or the second tool advancement device 204 may be configured to rotate. The first tool advancement device 202 and/or the second tool advancement device 204 may be configured to rotate around an axis that may extend along a length of the endovascular tool 102. In some embodiments, the first tool advancement device 202 and/or the second tool advancement device 204 may be configured to rotate in order to rotate, roll, twist, and/or torque the endovascular tool 102 and/or the microcatheter 103.

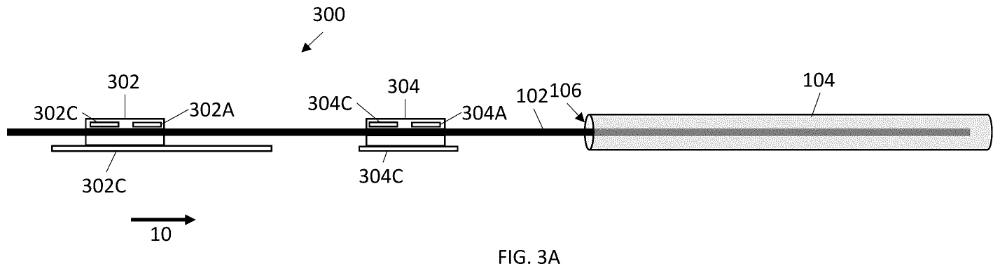

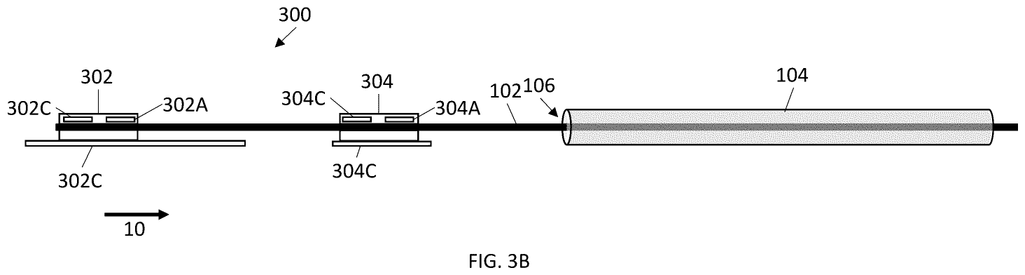

FIGS. 3A-3B show a system 300 for advancing an endovascular tool 102 through a patient lumen 104. Common features between the system 200 and the system 300 will not be described again but are incorporated here in their entirety. The system 300 may include a first tool advancement device 302 and a second tool advancement device 304. In some embodiments, the first tool advancement device 302 may include a rail system 302B and the second tool advancement device 304 may include a rail system 304B. In some embodiments, the first tool advancement device 302 includes an advancement mechanism 302C and the second tool advancement device 304 may include an advancement mechanism 304C. In some embodiments, the advancement mechanisms 302C, 304C may be configured to move (e.g., open/close) between an open configuration and a closed configuration. In some embodiments, the first tool advancement device 302 may include an actuator 302A configured to move the advancement mechanism 302C between the open configuration and the closed configuration, and the second tool advancement device 304 may include an actuator 304A configured to move the advancement mechanism 304C between the open configuration and the closed configuration.

In some embodiments, when the advancement mechanism 302C of the first tool advancement device 302 is in the open configuration, the advancement mechanism 302C may not contact the endovascular tool 102. In some embodiments, when the advancement mechanism 304C of the second tool advancement device 304 is in the open configuration the advancement mechanism 204C may not contact the endovascular tool 102.

In some embodiments, as shown in FIGS. 3A and 3B, when the advancement mechanism 302C of the first tool advancement device 302 and/or the advancement mechanism 304C of the second tool advancement device 304 are in the closed configuration, the advancement mechanism 302C of the first tool advancement device 302 and/or the advancement mechanism 304C of the second tool advancement device 304 may contact the endovascular tool 102 and/or apply the force to the endovascular tool 102. In some embodiments, when the advancement mechanism 302C of the first tool advancement device 302 and/or the advancement mechanism 304C of the second tool advancement device 304 are in the closed configuration, the advancement mechanisms 302C, 304C may apply the axial force 10 to the endovascular tool 102 in order to advance the endovascular tool 102 through the patient lumen 104.

In some embodiments, after the advancement mechanism 302C of the first tool advancement device 302 and/or the advancement mechanism 304C of the second tool advancement device 304 advance the endovascular tool 102 through the patient lumen 104, the advancement mechanism 302C of the first tool advancement device 302 and/or the advancement mechanism 304C of the second tool advancement device 304 may move to the open configuration.

In some embodiments, the advancement mechanism 304C of the second tool advancement device 302 may be in the open configuration when the advancement mechanism 302C of the first tool advancement device 302 advances the endovascular tool 102 through the patient lumen 104. In some embodiments, after the advancement mechanism 302C of the first tool advancement device 302 advances the endovascular tool 102 through the patient lumen 104, the advancement mechanism 304C of the second tool advancement device 304 may move to the closed configuration to apply the force to the endovascular tool 102 and/or the advancement mechanism 302C of the first tool advancement device 302 may move to the open configuration. In some embodiments, when the advancement mechanism 302C of the first tool advancement device 302 is in the open configuration the advancement mechanism 302C of the first tool advancement device 302 may move, translate, or otherwise reset in order to prepare to advance the endovascular tool 102 further though the patient lumen 104. In some embodiments, the force applied to the endovascular tool 102 by the advancement mechanism 304C of the second tool advancement device 304 may prevent or inhibit the advancement mechanism 302C of the first tool advancement device 302 from removing the endovascular tool 102 from the patient lumen 104.

In some embodiments, the advancement mechanism 302C of the first tool advancement device 302 and the advancement mechanism 304C of the second tool advancement device 304 may advance the endovascular tool 102 through the patient lumen 104. In some embodiments, both the advancement mechanism 302C of the first tool advancement device 302 and the advancement mechanism 304C of the second tool advancement device 304 may advance the endovascular tool 102 through the patient lumen 104 simultaneously. In some embodiments, the advancement mechanism 302C of the first tool advancement device 302 may advance the endovascular tool 102 through the patient lumen 104 when the advancement mechanism 304C of the second tool advancement device 304 prepares to advance the endovascular tool 102 through the patient lumen 104, and/or the advancement mechanism 304C of the second tool advancement device 304 may advance the endovascular tool 102 through the patient lumen 104 when the advancement mechanism 302C of the first tool advancement device 302 prepares to advance the endovascular tool 102 through the patient lumen 104 (i.e., the advancement mechanism 302C of the first tool advancement device 302 and the advancement mechanism 304C of the second tool advancement device 304 may advance the endovascular tool 102 through the patient lumen 104 in sequence or one after the other).

In some embodiments, when the advancement mechanism 302C of the first tool advancement device 302 and/or the advancement mechanism 304C of the second tool advancement device 304 advance the endovascular tool 102 through the patient lumen 104, the first tool advancement device 302 and/or the second tool advancement device 304 may remain stationary on the rail systems 302B, 304B (i.e., not translate along the rail systems 302B, 304B).

In some embodiments, the first tool advancement device 302 may be configured to rotate the advancement mechanism 302C and/or the actuator 302A may be configured to rotate the advancement mechanism 302C. In some embodiments, the second tool advancement device 304 may be configured to rotate the advancement mechanism 304C and/or the actuator 304A may be configured to rotate the advancement mechanism 304C. The advancement mechanism 302C of the first tool advancement device 302 and/or the advancement mechanism 304C of the second tool advancement device 304 may be configured to rotate around an axis that may extend along a length of the endovascular tool 102. In some embodiments, the advancement mechanism 302C of the first tool advancement device 302 and/or the advancement mechanism 304C of the second tool advancement device 304 may be configured to rotate in order to rotate, roll, twist, and/or torque the endovascular tool 102.

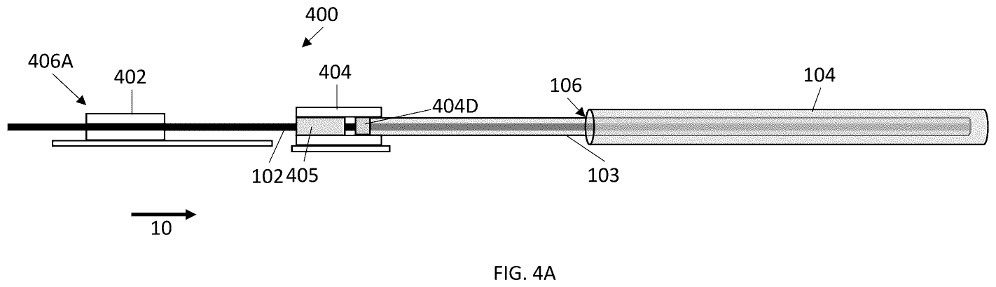

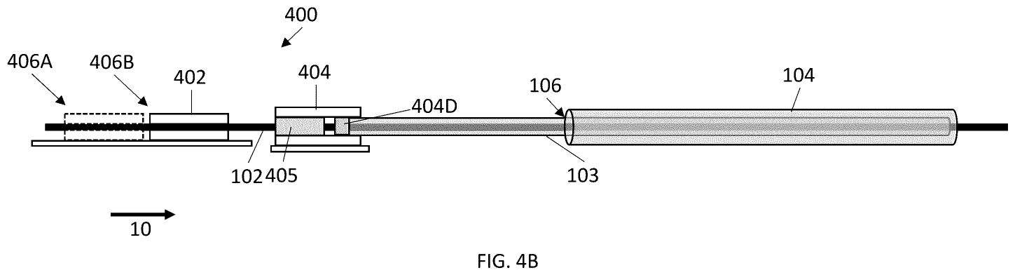

FIGS. 4A and 4B show a system 400 for advancing an endovascular tool 102 through a patient lumen. Common features between the systems 200, 300 and the system 400 will not be described again but are incorporated here in their entirety. The system 300 may include a first tool advancement device 402 and a second tool advancement device 404. In some embodiments, the second tool advancement device 404 may be coupled to a microcatheter 103. In some embodiments, the microcatheter 103 may include a luer attachment mechanism 103A at a proximal end 103B of the microcatheter 103. In some embodiments, the second tool advancement device 404 may be coupled to the luer attachment mechanism 103A of the microcatheter 103. In some embodiments, the second tool advancement device 404 may include a luer mate 404D, and the second tool advancement device 404 may be coupled to microcatheter 103 and/or the luer attachment mechanism 103A of the microcatheter 103 via the luer mate 404D. In some embodiments, the microcatheter 103 and/or the luer attachment mechanism 103A of the microcatheter may be screwed into or otherwise secured to the luer mate 404D.

In some embodiments, the system 400 may include a torquer 405. The torquer 405 may be coupled to the endovascular tool 102 and/or the endovascular tool 102 may extend through the torquer 405. The torquer 405 may be positioned proximal to the microcatheter 103 and/or the luer attachment mechanism 103A of the microcatheter 103.

In some embodiments, the second tool advancement device 404 may be coupled to the torquer 405 and/or the torquer 405 may be coupled to the second tool advancement device 404. In some embodiments, the second tool advancement device 404 may be configured to move (e.g., open/close) the torquer 405 between an open configuration and a closed configuration. In some embodiments, when the torquer 405 is in the open configuration, the torquer 405 may not contact the endovascular tool 102. Accordingly, the endovascular tool 102 may move relative to the torquer 405 so the endovascular tool 102 may advance through the torquer 405, the microcatheter 103, and/or a patient lumen 104. In some embodiments, when the torquer 405 is in the closed configuration, the torquer 405 may contact the endovascular tool 102. Accordingly, the torquer 405 may prevent the endovascular tool 102 from moving relative to the torquer 405 so the endovascular tool 102 may not advance through the torquer 405, the microcatheter 103, and/or the patient lumen 104.

In some embodiments, when the first tool advancement device 402 moves from a first position 406A to a second position 406B in order to advance the endovascular tool 102 through the patient lumen 104, the second tool advancement device 404 may move the torquer 405 to the open configuration so the endovascular tool 102 may advance through the patient lumen 104. In some embodiments, the second tool advancement device 404 may move the torquer 405 to the open configuration when the first tool advancement device 402 is positioned at the first position 406A. In some embodiments, the second tool advancement device 404 may move the torquer 405 to the open configuration before the first tool advancement device 402 moves from the first position 406A to the second position 406B.

In some embodiments, when the first tool advancement device 402 moves from the second position 406B to the first position 406A to prepare to advance the endovascular tool 102 through the patient lumen 104, the second tool advancement device may move the torquer 405 to the closed configuration in order to prevent or inhibit the first tool advancement device 402 from removing the endovascular tool 102 from the patient lumen 104. In some embodiments, the second tool advancement device 404 may move the torquer 405 to the closed configuration when the first tool advancement device 402 is positioned at the second position 406B. In some embodiments, the second tool advancement device 404 may move the torquer 405 to the closed configuration before the first tool advancement device 402 moves from the second position 406B to the first position 406A.

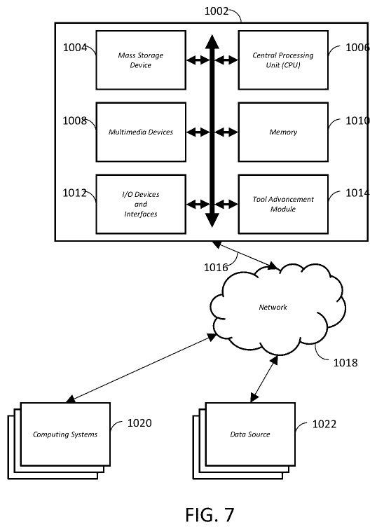

In some embodiments, a practitioner may control one or more functions, features, or components of the systems 200, 300, 400 via a computer system 1002, as described further below with reference to FIG. 7.

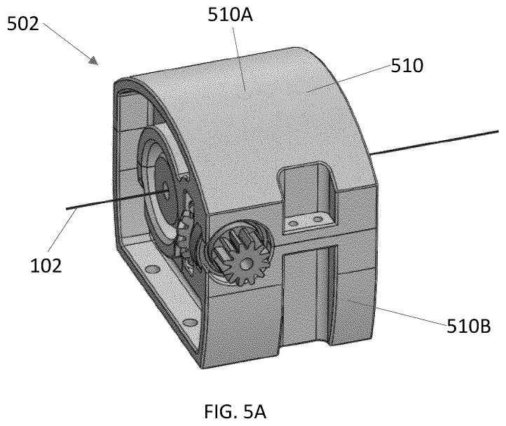

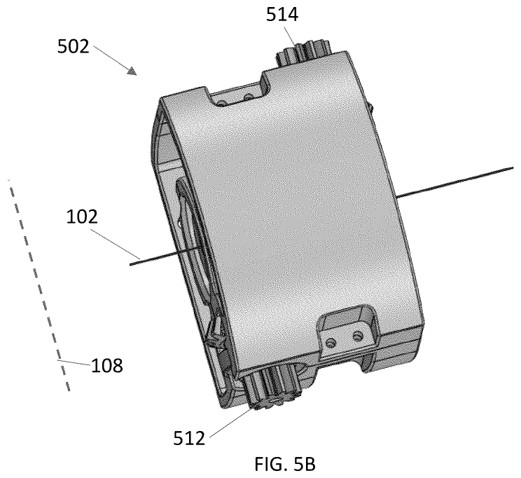

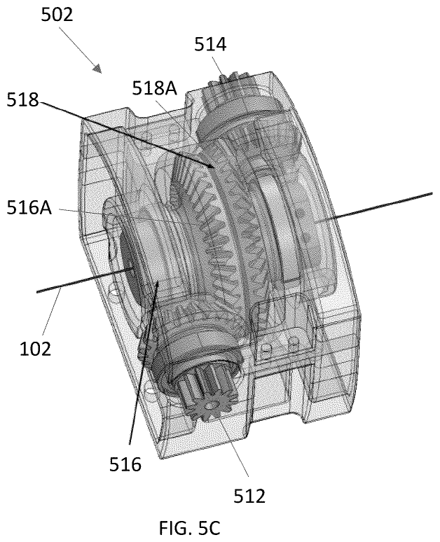

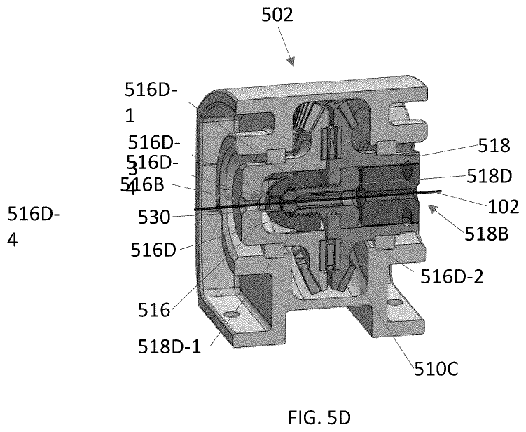

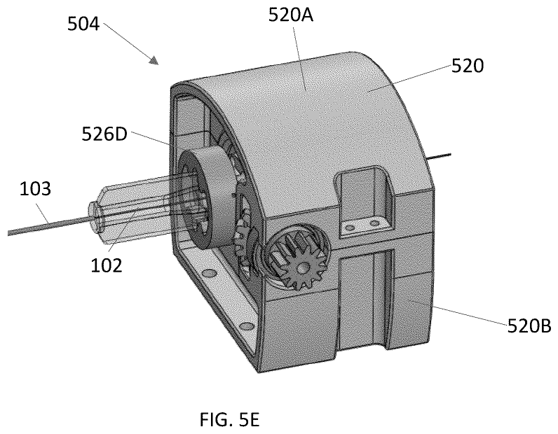

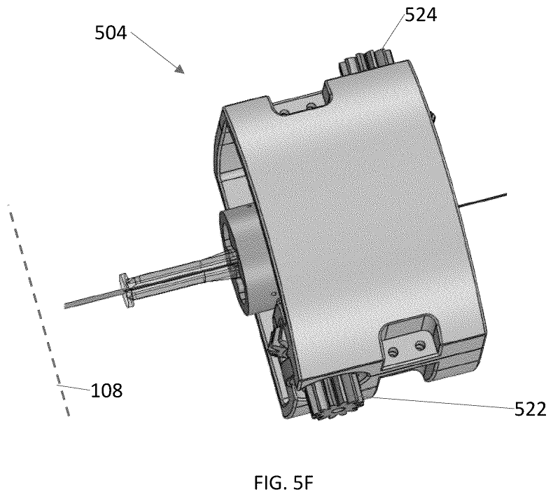

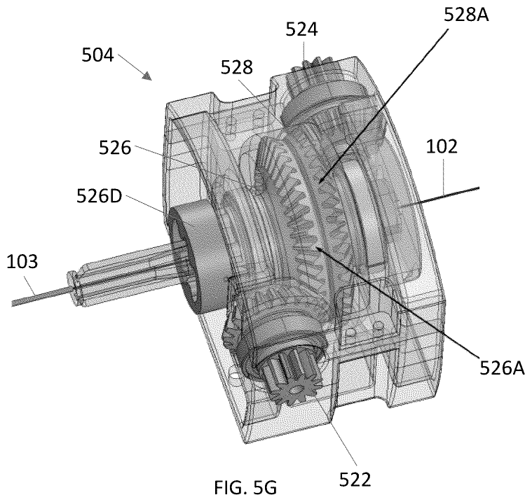

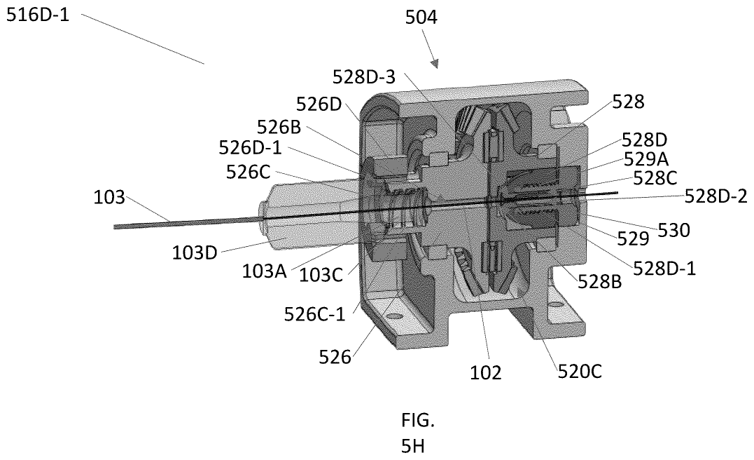

FIGS. 5A-5D show a first tool advancement device 502. In some embodiments, first tool advancement devices 202, 302, and 402 may include the first tool advancement device 502. In some embodiments, the first tool advancement device 502 may include a housing 510. In some embodiments, the housing 510 may include a top portion 510A and a bottom portion 510B. The top portion 510A may be removably coupled to the bottom portion 510B. In some embodiments, the housing 510 and/or the top portion 510A and the bottom portion 510B may include or define a recess 510C. The bottom portion 510B may be coupled to a rail system (not shown). The rail system may be configured to move the first tool advancement device 502 between a first position and a second position, as described above with reference to FIGS. 2A-2C and 4A-4B.

In some embodiments, as shown in FIG. 5B, the first tool advancement device 502 may include a first input 512 and a second input 514. In some embodiments, the first input 512 and/or the second input 514 may be remotely controlled via a computing system, a control system, and/or a controller. In some embodiments, the first input 512 and/or the second input 514 may be positioned between the top portion 510A of the housing 510 and the bottom portion 510B of the housing 510. In some embodiments, the top portion 510A may be uncoupled from the bottom portion 510B and the first input 512 and/or the second input 514 may be removed from the housing 510. In some embodiments, when the top portion 510A is coupled to the bottom portion 510B, the first input 512 and/or the second input 514 may rotate in a first direction (i.e., clockwise) and/or a second direction opposite the first direction (i.e., counterclockwise).

In some embodiments, as shown in FIG. 5C, the first input 512 may be configured interact with a first clamping device 516 and/or the second input 514 may be configured interact with a second clamping device 518. The first clamping device 516 may include a gear 516A and/or the second actuator 518 may include a gear 518A. In some embodiments, the gear 516A of the first clamping device 516 may interact with the first input 512 so that when the first input 512 is rotated, the first clamping device 516 may rotate. In some embodiments, the first input 512 may include a gear 512A configured to interact with the gear 516A of the first clamping device 516. In some embodiments, the gear 518A of the second clamping device 518 may interact with the second input 514 so that when the second input 514 is rotated, the second clamping device 518 may rotate. In some embodiments, the second input 514 may include a gear 514A configured to interact with the gear 518A of the second clamping device 518.

In some embodiments, as shown in FIG. 5D, the first clamping device 516 may include an opening 516B and/or the second clamping device 518 may include an opening 518B. In some embodiments, the opening 516B of the first clamping device 516 may be positioned at the center of the first clamping device 516 and/or the opening 518B of the second clamping device 518 may be positioned at the center of the second clamping device 518. In some embodiments, the endovascular tool 102 may be inserted through the opening 516B of the first clamping device 516 and/or the opening 518B of the second clamping device 518.

In some embodiments, the first clamping device 516 and/or the second clamping device 518 may be positioned in the recess 510C of the housing 510. In some embodiments, the first clamping device 516 and/or the second clamping device 518 may be rotatably coupled to or positioned in the housing 510 so the first clamping device 516 and/or the second clamping device 518 may rotate.

In some embodiments, the first clamping device 516 may be positioned distal to the second clamping device 518 so the first clamping device 516 is positioned closer to the patient lumen 104 than the second clamping device 518. In some embodiments, the second clamping device 518 may be positioned proximal to the first clamping device 516 so the second clamping device 518 is further from the patient lumen 104 than the first clamping device 516.

In some embodiments, the first clamping device 516 may include a recess 516C. The recess 516C may be configured to receive and/or hold a first collet clamp 516D. In some embodiments, the first collet clamp 516D may be configured to translate proximally and/or distally in the recess 516C. In some embodiments, the second clamping device 518 may include a recess 518C. The recess 518C may be configured to receive and/or hold a second collet clamp 518D. In some embodiments, the second collet clamp 518D may be configured to translate or move proximally and/or distally in the recess 518C. In some embodiments, the first collet clamp 516D may include a recess 516D-2 configured to receive the second collet clamp 518D. In some embodiments, the recess 516D-2 may include internal threading 516D-1 (i.e., female threading) and the second collet clamp 518D may include external threading 518D-1 (i.e., male threading).

In some embodiments, the first clamping device 516 and/or the second clamping device 518 may be positioned such that the second collet clamp 518D may extend from the recess 518C of the second clamping device 518 and into the first collet clamp 516D. In some embodiments, the external threading 518D-1 of the second collet clamp 518D may interact with the internal threading 516D-1 of the first collet clamp 516D to threadably couple the second collet clamp 518D to the first collet clamp 516D. In some embodiments, the second collet clamp 518D may translate towards (i.e., into the recess 516D-2) and/or away from (i.e., out of the recess 516D-2) the first collet clamp 516D when the second collet clamp 518D is rotated relative to the first collet clamp 516D.

In some embodiments, the first collet clamp 516D may be coupled to the first clamping device 516 so the first collet clamp 516D may rotate around the endovascular tool 102 when the first clamping device 516 rotates around the endovascular tool 102. In some embodiments, the second collet clamp 518D may be coupled to the second clamping device 518 so the second collet clamp 518D may rotate around the endovascular tool 102 when the second clamping device 518 rotates around the endovascular tool 102.

In some embodiments, the first clamping device 516 may be rotated relative to the second clamping device 518 and/or the second clamping device 518 may be rotated relative to the first clamping device 516. In some embodiments, the first clamping device 516 and the second clamping device 518 may be rotate in opposite direction. In some embodiments, when the first clamping device 516 is rotated in a first direction and the second clamping device 518 is rotated in a second direction, the internal threading 516D-1 of the first collet clamp 516D and the external threading 518D-1 of the second collet clamp 518 may translate the second collet clamp 518 towards the first collet clamp 516D. In some embodiments, when the first clamping device 516 is rotated in the second direction and the second clamping device 518 is rotated in the first direction, the second collet clamp 518D may translate away from the first collet clamp 516D.

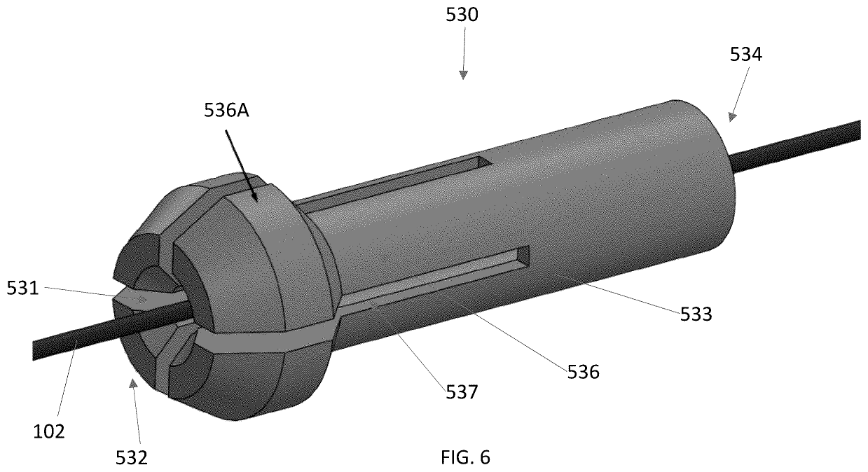

In some embodiments, the first tool advancement device 502 may include a collet 530, as shown in FIG. 6. In some embodiments, the collet 530 may be positioned in the second collet clamp 518D so the second collet clamp 518D and the collet 530 may be positioned concentrically around the endovascular tool 102.

In some embodiments, the collet 530 may include a cylindrical shape, a rectangular prism shape, and/or any other shape. The collet 530 may include distal end 532 and a proximal end 534. In some embodiments, the collet 530 may include an opening 531 extending through the collet 530 from the distal end 532 to the proximal end 534. In some embodiments, the endovascular tool 102 may be inserted into and/or through the collet 530 and/or the opening 531.

In some embodiments, the collet 530 may include clamps 536. In some embodiments, the clamps 536 may be positioned at the distal end 532 of the collet 530. In some embodiments, the clamps 536 may be spaced around the opening 531 and the clamps 536 may be separated by cutouts 537. The cutouts 537 may extend from an outer surface 533 of the collet 530 to the opening 531 and/or the cutouts 537 may extend from the distal end 532 to a position 535 between the distal end 532 and the proximal end 534. In some embodiments, the cutouts 537 may allow the clamps 536 to radially flex towards each other and/or away from each other.

In some embodiments, the clamps 536 may define the opening 531 at the distal end 532. In some embodiments, when the clamps 536 flex towards each other, the opening 531 at the distal end 532 may become smaller and/or the clamps 536 may contact the endovascular tool 102 inserted through the opening 531. Accordingly, when the clamps 536 contact the endovascular tool 102, the first tool advancement device 502 may be in the closed configuration, and the clamps 536 and/or the collet 530 may prevent or inhibit the endovascular tool 102 from translating through the opening 531 relative to the collet 530.

In some embodiments, a portion 536A of the clamps 536 positioned at the distal end 532 may extend radially outward from the collet 530. In some embodiments, as shown in FIG. 5C, when the second collet clamp 518D translates towards the first collet clamp 516D, the second collet clamp 518D may translate or move the collet 530 towards the first collet clamp 516D. In some embodiments, the first collet clamp 516D may include an opening 516D-3 at a distal end 516D-4 of the first collet clamp 516D. In some embodiments, the opening 516D-3 may be smaller than the recess 516D-2. Accordingly, when the collet 530 translates towards the first collet clamp 516D, the clamps 536 may flex towards each other in order to fit into the opening 516D-3 and/or the first collet clamp 516D may apply a force to the portion 536A of the clamps 536 so the clamps 536 may flex towards each other. In some embodiments, the second collet clamp 518D may apply a force to the portion 536A of the clamps 536 so the clamps 536 may flex towards each other. In some embodiments, the first collet clamp 516D and the second collet clamp 518D may apply a force to the portion 536A of the clamps 536.

In some embodiments, the first clamping device 516 and the second clamping device 518 may be rotated in the same direction around the endovascular tool 102. In some embodiments, the first clamping device 516 and the second clamping device 518 may both be rotated in the first direction or the first clamping device 516 and the second clamping device 518 may both be rotated in the second direction. In some embodiments, when the first clamping device 516 and the second clamping device 518 are rotated in the same direction, the first clamping device 516 may rotate the first collet clamp 516D and the second clamping device 518 may rotate the second collet clamp 518D. Accordingly, the first collet clamp 516D and the second collet clamp 518D may rotate around the endovascular tool 102 and the second collet clamp 518D may not translate towards or away from the first collet clamp 516D. In some embodiments, if the collet 530 is in the open configuration, the collet 530 may rotate around the endovascular tool 102. In some embodiments, if the collet 530 is in the closed configuration, the collet 530 may rotate, roll, twist, and/or torque the endovascular tool 102.

In some embodiments, the first input 512 may be positioned perpendicular or substantially perpendicular to the first clamping device 516. In some embodiments, as shown in FIG. 5C, the first input 512 may be positioned or configured to rotate around an axis 108 perpendicular to the endovascular tool 102 and/or the first clamping device 516 may be positioned or configured to rotate around the endovascular tool 102, as described above. In some embodiments, the second input 514 may be positioned perpendicular or substantially perpendicular to the second clamping device 518. In some embodiments, as shown in FIG. 5C, the second input 514 may be positioned or configured to rotate around the axis 108 perpendicular to the endovascular tool 102 and/or the second clamping device 518 may be positioned or configured to rotate around the endovascular tool 102, as described above.

FIGS. 5E-5H show a second tool advancement device 504. In some embodiments, the second tool advancement devices 204, 304, and 404 may include the second tool advancement device 504. In some embodiments, the second tool advancement device 504 may include a housing 520. In some embodiments, the housing 520 may include a top portion 520A and a bottom portion 520B. The top portion 520A may be removably coupled to the bottom portion 520B. In some embodiments, the housing 520 and/or the top portion 520A and the bottom portion 520B may include or define a recess 520C. The bottom portion 520B may be coupled to rail system (not shown). The rail system may be configured to move the second tool advancement device 504 between a third position and a fourth positioned, as described above with reference to FIGS. 2A-2C and 4A-4B.

In some embodiments, as shown in FIG. 5F, the second tool advancement device 504 may include a first input 522 and a second input 524. In some embodiments, the first input 522 and/or the second input 522 may be remotely controlled via a computing system, a control system, and/or a controller. In some embodiments, the first input 522 and/or the second input 524 may be positioned between the top portion 520A of the housing 520 and the bottom portion 520B of the housing 520. In some embodiments, the top portion 520A may be uncoupled from the bottom portion 520B and the first input 522 and/or the second input 524 may be removed from the housing 510. In some embodiments, when the top portion 520A is coupled to the bottom portion 520B, the first input 522 and/or the second input 514 may rotate in a first direction (i.e., clockwise) and/or a second direction opposite the first direction (i.e., counterclockwise).

In some embodiments, as shown in FIG. 5G, the first input 522 may be configured to interact with a first clamping device 526 and/or the second input 524 may be configured to interact with a second clamping device 528. The first clamping device 526 may include a gear 526A and/or the second actuator 528 may include a gear 528A. In some embodiments, the gear 526A of the first clamping device 526 may interact with the first input 522 so that when the first input 522 is rotated, the first clamping device 526 may rotate. In some embodiments, the first input 522 may include a gear 522A configured to interact with the gear 526A of the first clamping device 526. In some embodiments, the gear 528A of the second clamping device 528 may interact with the second input 524 so that when the second input is rotated 524, the second clamping device 528 may rotate. In some embodiments, the second input 524 may include a gear 524A configured to interact with the gear 528A of the second clamping device 528.

In some embodiments, as shown in FIG. 5H, the first clamping device 526 may include an opening 526B and/or the second clamping device 528 may include an opening 528B. In some embodiments, the opening 526B of the first clamping device 526 may be positioned at the center of the first clamping device 526 and/or the opening 528B may of the second clamping device 528 may be positioned at the center of the second clamping device 528. In some embodiments, the endovascular tool 102 may be inserted through the opening 526B of the first clamping device 526 and/or the opening 528B of the second clamping device 528.

In some embodiments, the first clamping device 526 and/or the second clamping device 528 may be positioned in the recess 520C of the housing 520. In some embodiments, the first clamping device 526 and/or the second clamping device 528 may be rotatably coupled to or positioned in the housing 520 so the first clamping device 526 and/or the second clamping device 528 may rotate.

In some embodiments, the first clamping device 526 may be positioned distal to the second clamping device 528 so the first clamping device 526 is positioned closer to the patient lumen than the second clamping device 528. In some embodiments, the second clamping device 528 may be positioned proximal to the first clamping device 526 so the second clamping device 528 is further from the patient lumen 104 than the first clamping device 526.

In some embodiments, the first clamping device 526 may include a recess 526C. In some embodiments, the first clamping device 526 and/or the recess 526C may be configured to extend distally from the housing 520. The recess 526C may be configured to receive the microcatheter 103 and/or the luer attachment mechanism 103A of the microcatheter 103. In some embodiments, the microcatheter 103 and/or the luer attachment mechanism 103A may be coupled to the first clamping device 526 and/or the recess 526C. In some embodiments, the endovascular tool 102 may be inserted through the microcatheter 103 when the microcatheter 103 and/or the luer attachment mechanism 103A are coupled to the first clamping device 526 and/or the recess 526C In some embodiments, the recess 526C may include internal threading 526C-1 (i.e., female threading). In some embodiments, the internal threading 526C-1 may be configured to interact with external threading 103C (i.e., male threading) of the luer attachment mechanism 103A. In some embodiments, the internal threading 526C-1 and/or the external threading 103C may couple the luer attachment mechanism 103A and/or the microcatheter 103 to the recess 526C and/or the first clamping device 526.

In some embodiments, the first clamping device 526 may include a microcatheter securement device 526D. In some embodiments, the microcatheter securement device 526D may include a ring shape. In some embodiments, the microcatheter securement device 526D may include one or more indentations 526D-1. In some embodiments, the microcatheter securement device 526D may be slidably coupled to the first clamping device 526. In some embodiments, the microcatheter securement device 526D may be coupled to the first clamping device 526 when the microcatheter 103 is coupled to the first clamping device 526. In some embodiments, the microcatheter securement device 526D may inserted onto the first clamping device 526. In some embodiments, the one or more indentations 526D-1 may be positioned so the one or more indentations 526D-1 are inserted over flanges 103D of the microcatheter 103.

In some embodiments, when the first clamping device 526 rotates in a first direction, the first clamping device 526 may rotate the microcatheter 103 in the first direction and/or when the first clamping device 526 rotates in a second direction, the first clamping device 526 may rotate the microcatheter 103 in the second direction. In some embodiments, when the first clamping device 526 rotates the first clamping device 526 may rotate the microcatheter securement device 526D. In some embodiments, the microcatheter securement device 526D may apply a rotational force to a portion of the flanges 103D in the one or more indentations 526D-1 to rotate the flanges 103D and/or the microcatheter 103.

In some embodiments, the second clamping device 528 may include a recess 528C. In some embodiments, the recess 528C may be configured to receive and/or hold a first collet clamp 528D. In some embodiments, the first collet clamp 528D may include a recess 528D-2 with internal threading 528D-1. In some embodiments, the recess 528D-2 of the first collet clamp 528D may be configured to receive a second collet clamp 529. In some embodiments, the second collet clamp 529 may include external threading 529A. In some embodiments, the second collet clamp 529 may be positioned in the recess 520C of the housing 520. In some embodiments, the second collet clamp 529 may be coupled to or fixed to the housing 510. In some embodiments, the second collet clamp 529 may be positioned proximal to the first clamping device 526 and the second clamping device 528.

In some embodiments, the second collet clamp 529 may be positioned so the second collet clamp 529 may extend into the recess 528C of the second clamping device 528 and/or the first collet clamp 528D. In some embodiments, the external threading 529A of the second collet clamp 529 may interact with the internal threading 528D-1 of the first collet clamp 528D to threadably couple the second collet clamp 529 to the first collet clamp 528D. In some embodiments, when the first collet clamp 528D rotates in a first direction, the first collet clamp 528D may translate towards the second collet clamp 529 (i.e., the second collet clamp 529 may translate into the recess 528D-2).

In some embodiments, the second tool advancement device 504 may include a collet 530, as described above with reference to FIG. 6. In some embodiments, the collet 530 may be positioned in the second collet clamp 529 so the second collet clamp 529 and the collet 530 may be positioned concentrically around the endovascular tool 102. In some embodiments, the second clamping device 528 may be rotated around the endovascular tool 102. In some embodiments, when the second clamping device 528 is rotated in a first direction, the first collet clamp 528D may rotate relative to the second collet clamp 529 in the first direction. Accordingly, the internal threading 528D-1 of the first collet clamp 528D and the external threading 529A of the second collet clamp 529 may translate the first collet clamp 528D towards the second collet clamp 529. When the first collet clamp 528D translates towards the second collet clamp 529, the first collet clamp 528D may translate toward the collet 530. In some embodiments, the first collet clamp 528D may include an opening 528D-3 at a distal end 528D-4 of the first collet clamp 528D. In some embodiments, the opening 528D-3 may be smaller than the recess 528D-2. Accordingly, when the first collet clamp 528D translates towards the collet 530, the clamps 536 of the collet 530 may flex towards each other in order to fit into the opening 528D-2 and/or the first collet clamp 528D may apply a force to the portion 536A of the clamps 536 so the clamps 536 may flex towards each other. In some embodiments, the second collet clamp 529 may apply a force to the portion 536A of the clamps 536 so the clamps 536 may flex towards each other (i.e., the closed configuration) so the clamps 56 contact the endovascular tool 102. In some embodiments, the first collet clamp 5528D and the second collet clamp 529 may apply a force to the portion 536A of the clamps 536.

In some embodiments, when the second clamping device 528 is rotated in a second direction opposite the first direction, the first collet clamp 528D may rotate relative to the second collet clamp 529 in the second direction. Accordingly, the internal threading 528D-1 of the first collet clamp 528D and the external threading 529A of the second collet clamp 529 may translate the first collet clamp 528D away from the second collet clamp 529. When the first collet clamp 528D translates away from the second collet clamp 529, the first collet clamp 528D may translate away from the collet 530. Accordingly, the first collet clamp 528D and/or the second collet clamp 529 may apply a smaller force or no force to the portion 536A of the clamps 536, and the clamps 536 may flex away from each other (i.e., the open configuration) so the clamps 536 do not contact the endovascular tool 102.

In some embodiments, the first input 522 may be positioned perpendicular or substantially perpendicular to the first clamping device 526. In some embodiments, FIG. 5G, the first input device may be positioned or configured to rotate around the axis 108 perpendicular to the endovascular tool 102 and/or the first clamping device 526. In some embodiments, the second input 524 may be positioned perpendicular or substantially perpendicular to the second clamping device 528. In some embodiments, as shown in FIG. 5G, the second input 524 may be positioned or configured to rotate around the axis 108 perpendicular to the endovascular tool 102 and/or the second clamping device 528 may be positioned or configured to rotate around the endovascular tool 102, as described above.

### Computer System

In some embodiments, the systems, processes, and methods described herein are implemented using a computing system, such as the one illustrated in FIG. 7. The example computer system 1002 is in communication with one or more computing systems 1020 and/or one or more data sources 1022 via one or more networks 1018. While FIG. 7 illustrates an embodiment of a computer system 1002, it is recognized that the functionality provided for in the components and modules of computer system 1002 can be combined into fewer components and modules, or further separated into additional components and modules.

The computer system 1002 can comprise a tool advancement module 1014 that carries out the functions, methods, acts, and/or processes described herein. The module 1014 is executed on the computer system 1002 by a central processing unit (CPU) 1006 discussed further below.

In general the word “module,” as used herein, refers to logic embodied in hardware or firmware or to a collection of software instructions, having entry and exit points. Modules are written in a program language, such as JAVA, C, C++, and/or the like. Software modules can be compiled or linked into an executable program, installed in a dynamic link library, or can be written in an interpreted language such as BASIC, PERL, LAU, PHP, or Python and/or any such languages. Software modules can be called from other modules or from themselves, and/or can be invoked in response to detected events or interruptions. Modules implemented in hardware include connected logic units such as gates and flip-flops, and/or can include programmable units, such as programmable gate arrays and/or processors.

Generally, the modules described herein refer to logical modules that can be combined with other modules or divided into sub-modules despite their physical organization or storage. The modules are executed by one or more computing systems and can be stored on or within any suitable computer readable medium, or implemented in-whole or in-part within special designed hardware or firmware. Not all calculations, analysis, and/or optimization require the use of computer systems, though any of the above-described methods, calculations, processes, or analyses can be facilitated through the use of computers. Further, in some embodiments, process blocks described herein can be altered, rearranged, combined, and/or omitted.

The computer system 1002 includes one or more CPU 1006, which can comprise a microprocessor. The computer system 1002 further includes a physical memory 1010, such as random access memory (RAM) for temporary storage of information, a read only memory (ROM) for permanent storage of information, and a mass storage device 1004, such as a backing store, hard drive, rotating magnetic disks, solid state disks (SSD), flash memory, phase-change memory (PCM), 3D XPoint memory, diskette, or optical media storage device. Alternatively, the mass storage device can be implemented in an array of servers. Typically, the components of the computer system 1002 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 1002 includes one or more input/output (I/O) devices and interfaces 1012, such as a keyboard, mouse, touch pad, and printer. The I/O devices and interfaces 1012 can include one or more display devices, such as a monitor, that allows the visual presentation of data to a user. More particularly, a display device provides for the presentation of GUIs as application software data, and multi-media presentations, for example. The I/O devices and interfaces 1012 can also provide a communications interface to various external devices. The computer system 1002 can comprise one or more multi-media devices 1008, such as speakers, video cards, graphics accelerators, and microphones, for example.

### Computing System Device/Operating System

The computer system 1002 can run on a variety of computing devices, such as a server, a Windows server, a Structure Query Language server, a Unix Server, a personal computer, a laptop computer, and so forth. In other embodiments, the computer system 1002 can run on a cluster computer system, a mainframe computer system and/or other computing system suitable for controlling and/or communicating with large databases, performing high volume transaction processing, and generating reports from large databases. The computer system 1002 is generally controlled and coordinated by an operating system software, such as z/OS, Windows, Linux, UNIX, BSD, PHP, SunOS, Solaris, MacOS, ICloud services or other compatible operating systems, including proprietary operating systems. Operating systems control and schedule computer processes for execution, perform memory management, provide file system, networking, and I/O services, and provide a user interface, such as a graphical user interface (GUI), among other things.

### Network

The computer system 1002 illustrated in FIG. 7 is coupled to a network 1018, such as a LAN, WAN, or the Internet via a communication link 1016 (wired, wireless, or a combination thereof). Network 1018 communicates with various computing devices and/or other electronic devices. Network 1018 is communicating with one or more computing systems 1020 and one or more data sources 1022. The tool advancement module 1014 can access or can be accessed by computing systems 1020 and/or data sources 1022 through a web-enabled user access point. Connections can be a direct physical connection, a virtual connection, and other connection type. The web-enabled user access point can comprise a browser module that uses text, graphics, audio, video, and other media to present data and to allow interaction with data via the network 1018.

The output module can be implemented as a combination of an all-points addressable display such as a cathode ray tube (CRT), a liquid crystal display (LCD), a plasma display, or other types and/or combinations of displays. The output module can be implemented to communicate with input devices 1012 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module can communicate with a set of input and output devices to receive signals from the user.

### Other Systems

The computer system 1002 can include one or more internal and/or external data sources (for example, data sources 1022). In some embodiments, one or more of the data repositories and the data sources described above can be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 1002 can also access one or more databases 1022. The databases 1022 can be stored in a database or data repository. The computer system 1002 can access the one or more databases 1022 through a network 1018 or can directly access the database or data repository through I/O devices and interfaces 1012. The data repository storing the one or more databases 1022 can reside within the computer system 1002.

### URLs and Cookies

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Doman Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

### Embodiments

It will now be evident to those skilled in the art that there has been described herein methods, systems, and devices for improved routing of catheters and other devices to targeted anatomical locations using robotically controlled assemblies. Although the inventions hereof have been described by way of several embodiments, it will be evident that other adaptations and modifications can be employed without departing from the spirit and scope thereof. The terms and expressions employed herein have been used as terms of description and not of limitation; and thus, there is no intent of excluding equivalents, but on the contrary it is intended to cover any and all equivalents that may be employed without departing from the spirit and scope of the inventions.

While the disclosure has been described with reference to certain embodiments, it will be understood that various changes may be made, and equivalents may be substituted for elements thereof without departing from the scope of the disclosure. In addition, many modifications will be appreciated to adapt a particular instrument, situation, or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above but should be determined only by a fair reading of the claims that follow.

While the embodiments disclosed herein are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “advancing a catheter or microcatheter” or “advancing one portion of the device (e.g., linearly) relative to another portion of the device to rotate the distal end of the device” include instructing advancing a catheter” or “instructing advancing one portion of the device,” respectively. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

What is claimed is:

1. A system for advancing a guidewire through a patient lumen, the system comprising:
a first advancement device configured to move between a first position and a second position a distance from the first position, wherein the first position is proximal to the second position, the first advancement device comprising:
a first clamp configured to move between an open configuration and a closed configuration,
wherein the first clamp is configured to contact the guidewire when the first clamp is in the closed configuration;
a second advancement device positioned between the first advancement device and an access point to the patient lumen, the second advancement device comprising:
a second clamp configured to move between an open configuration and a closed configuration,
wherein the second clamp is configured to contact the guidewire when the second clamp is in the closed configuration;
wherein the first advancement device is configured to move from the first position to the second position when the first clamp is in the closed configuration to advance the guidewire the distance through the patient lumen.

2. The system of claim 1, wherein the first advancement device is coupled to a rail system, wherein the rail system is configured to move the first advancement device between the first position and the second position.

3. The system of claim 1, wherein the second advancement device is coupled to a microcatheter, and wherein the guidewire is inserted through the microcatheter.

4. The system of claim 3, wherein the second advancement device is configured to move between a third position and a fourth position the distance from the third position, wherein the third position is proximal to the fourth position, and wherein second advancement device is configured to move from the third position to the fourth position to advance the microcatheter through the patient lumen.

5. The system of claim 1, wherein the second clamp is in the open configuration when the first advancement device moves from the first position to the second position.

6. The system of claim 5, wherein the first clamp is configured to move from the closed configuration to the open configuration after the first advancement device moves from the first position to the second position, and wherein the second clamp is configured to move from the open configuration to the closed configuration after the first advancement device moves from the first position to the second position.

7. The system of claim 6, wherein the first advancement device is configured to move from the second position to the first position when the first clamp is in the open configuration.

8. The system of claim 1, wherein the distance is between about 20 mm and about 60 mm.

9. A tool advancement device comprising:
a housing comprising a first recess
a first gear positioned in the first recess, the first gear configured to rotate in a first direction around a guidewire and a second direction around the guidewire, the second direction opposite the first direction, the first gear comprising:
a second recess;
a first collet clamp positioned in the second recess, the first collet clamp configured to rotate with the first gear, the first collet clamp comprising a third recess, the third recess comprising internal threading;
a second gear positioned in the first recess, the second gear configured to rotate in the first direction and the second direction;
a first input configured to rotate the first gear in the first direction and the second direction;
a second input configured to rotate the second gear in the first direction and the second direction; and
a second collet clamp comprising a fourth recess, and external threading configured to interact with the internal threading of the first collet clamp, wherein the second collet clamp is configured to extend into the third recess of the first collet clamp;
a collet positioned in the fourth recess of the second collet clamp, the collet configured to move between an open configuration and a closed configuration, wherein the collet contact a guidewire when the collet is in the closed configuration
wherein the internal threading and the external threading are configured to translate the first collet clamp towards the second collet clamp to move the collet to the closed configuration when the first gear is rotated relative to the second gear in the first direction, and wherein the internal threading and the external threading are configured to translate the first collet clamp away from the second collet clamp to move the collet to the open configuration when the first gear is rotated relative to the second gear in the second direction.

10. The tool advancement device of claim 9, wherein the second gear is positioned proximal to the first gear, and wherein the second collet clamp is positioned in a fifth recess of the second gear.

11. The tool advancement device of claim 10, wherein when the collet is in the closed configuration, the collet is configured to rotate the guidewire when the first gear and the second gear are rotated in the first direction or the second direction.

12. The tool advancement device of claim 10, wherein the first gear is rotated in the first direction and the second gear is rotated in the second direction to rotate the first gear relative to the second gear in the first direction, and wherein the wherein the first gear is rotated in the second direction and the second gear is rotated in the first direction to rotate the first gear relative to the second gear in the second direction.

13. The tool advancement device of claim 9, wherein the second collet clamp is coupled to the housing.

14. The tool advancement device of claim 9, wherein the second gear is positioned distal to the first gear, and wherein the second gear comprises a fifth recess, the fifth recess configured extend distally from the first recess of the housing, wherein the fifth recess is configured to receive a microcatheter positioned over the guidewire.

15. The tool advancement device of claim 14, wherein the second gear is configured to rotate the microcatheter in the first direction when the second gear rotates in the first direction, and wherein the second gear is configured to rotate the microcatheter is the second direction when the second gear rotates in the second direction.

16. A method for advancing a guidewire through a patient lumen, the method comprising the steps of:
inserting a guidewire through a first advancement device and a second advancement device, wherein the second advancement device is positioned between the first advancement device and an access point to a patient lumen;
moving the first advancement device to a closed configuration, wherein the first advancement device contacts the guidewire when the first advancement device is in the closed configuration;
moving the first advancement device from a first position to a second position to advance the guidewire a distance through the patient lumen, wherein the second position is the distance from the first position between the first position and the access point.

17. The method of claim 16 further comprising the steps of:
moving the first advancement device to an open configuration after the first tool advancement device moves from the first position to the second position so the first tool advancement device does not contact the guidewire;
moving the second advancement device to the closed configuration;
moving the first advancement device from the second position to the first position;
moving the first advancement device to the closed configuration and the second advancement device to the open configuration.

18. The method of claim 17, wherein the guidewire is inserted through a microcatheter, and the second advancement device is coupled to the microcatheter.

19. The method of claim 18, further comprising moving the second advancement device from a third position to a fourth position to advance the microcatheter through the patient lumen, wherein the fourth position in between the third position and the access point.

20. The method of claim 16, wherein the distance is between about 20 mm and about 60 mm.
